(19) United States
(12) Patent Application Publication
(10) Pub. No.: US 2003/0143108 A1
(43) Pub. Date: Jul. 31, 2003

(54) Apparatus and a method for decontamination

(76) Inventor: Eric Wasinger, San Antonio, TX (US)

Correspondence Address: John Lezdey & Associates, 4625 East Bay Drive, Suite 302, Clearwater, FL, 33764, US

(21) Appl. No.: 10/060,668

(22) Filed: Jan. 30, 2002

(51) Int. Cl. A01N 1/00; B01J 19/08

(52) U.S. Cl. 422/28; 422/33; 422/34; 422/186.07

(57) ABSTRACT

This invention relates to a method and apparatus for using ozone to disinfect and decontaminate infectious microbial agents including mold in a building or container. This invention contemplates treating biological and viral agents with ozone containing gases.

### FIELD OF THE INVENTION

This invention relates to a method and apparatus for using ozone to disinfect and decontaminate infectious microbial agents, especially anthrax (Bacillus anthracis) and smallpox (variola; variola minor and variola major).

### DESCRIPTION OF THE PRIOR ART

Strong oxidizing agents such as hypochlorite solutions have been used as antimicrobial agents.

Industry and the US military have long sought an alternative method for disinfecting and decontaminating infectious microbial agents such as anthrax and smallpox. This particular topic has now come to the forefront because of bioterrorist activities in the United States since Sep. 11, 2001.

Treatment of anthrax in humans is effective with antibiotics provided treatment is initiated at an early stage. Treatment of smallpox is done by prior vaccination with the smallpox vaccine but us only effective for about three years.

At the present time the use of strong liquid oxidizing agents work well in disinfecting and decontaminating specific items. However, it is impossible to treat the contents of a building effectively since the contents of a building such as furniture, fixtures, electronic equipment, paper documents, and environmental duct work afford excellent hiding places for biological and viral agents. Therefore, it is very difficult or impossible to remove airborne biological and viral agents by merely cleaning the surface.

Another problem with handling strong liquid oxidizers is the hazards in transportation and use. Human exposure to high concentrations of these liquid oxidizers can result in serious dermatological and pulmonary injuries.

Another problem with using liquid oxidizers is that certain contents of a building require total saturation to be effective. An example of this would be the contamination of a computer keyboard, the mere act of wiping the keyboard with a sponge containing a liquid oxidizer, such as bleach, would most likely kills agents present on the surface of the keys. However, the agent(s) that have migrated to the spaces between the keys or under the keyboard panel would not be affected, This principal can be applied to almost every item making up the contents of a building. It is impractical to saturate all the contents of a building with a liquid oxidizing agent. The liquid oxidizers have been misted into such buildings, namely, those contaminated with molds that sicken people, especially asthmatics.

Another method for disinfecting and decontaminating of these agents is the use of ultraviolet light. Ultraviolet light is better for disinfecting and decontaminating airborne agents than liquid oxidizers. However, the problem with using UV light is that it is only effective if the light can illuminate the microbial agent. Agents that have migrated into the duct work of a building would not be affected, nor would agents in non-illuminated areas of a building.

Ozone has been used to disinfect and decontaminate a broad variety of pathogens such as bacteria, fungi viruses, parasites, and protozoans in aqueous systems.

Ozone is a powerful oxidizing agent. Ozone has 150% of the oxidation potential of chlorine and twice the oxidation potential of bromine. Ozone has been shown to be much more effective than chlorine with a reaction time up to 10 times faster. Unlike chlorine or hallogenated compounds, ozone has a negligible deleterious effect on the contents of a building because ozone breaks down into simple diatomic oxygen on reaction with an organic or inorganic agent. Moreover, ozone also readily self-destructs into simple diatomic oxygen due to its inherent instability.

Ozone breaks down complex biological agents into smaller molecules and eventually into H₂O and CO₂. Ozone kills biological agents by oxidizing the cell walls. When cells are exposed to ozone for a sufficient period of time, lying of cell walls occurs releasing the cytoplasm of the cells and causing destruction of cells.

A virus is approximately 1000 times smaller than bacteria and consists of a package of nucleic acid encased in a tough casing. The de-activation of virus by ozone is by oxidation of cell wall components and the nucleic acid.

### SUMMARY OF THE INVENTION

This disinfecting and decontaminating of infectious biological and viral agents including mold with liquid oxidizers is inefficient, cumbersome and destructive as it relates to preserving contents of a building.

This invention relates to a method for disinfecting and decontaminating infectious biological and viral agents, in particular anthrax and smallpox, that uses the efficient method of ozonation. A feature of the invention consists of using a known biological and/or viral agent placed into the treatment area prior to treatment as a control to determine the kill level of the ozonation process.

The principal objective of the invention is the provision of a method for efficiently disinfecting and decontaminating infectious biological and viral agents, especially anthrax and smallpox in a specific area, such as a building, a mailroom or an office.

It is a further object of the invention to provide a method that will disinfect and decontaminate infectious biological and viral agents, while at the same time preserve the integrity of the contents of said building.

Still another object of the invention is to provide a method of treating a specific item(s) in a pressure and/or airtight container that will disinfect and decontaminate the item while preserving the integrity of said item.

Yet another object of the invention is to provide a method of treating specific items, in particular letters and packages, in a pressure and/or airtight container that will disinfect and decontaminate the letters and packages while preserving the non-biological and non-viral contents.

Other objects and advantages of this invention will become apparent from the following description taken in connection with the accompanying drawing wherein is set forth by way of illustration and example an embodiment of this invention.

### BRIEF DESCRIPTION OF THE DRAWINGS

FIG. 1 is a schematic view of a method of decontaminating a building according to the present invention.

FIG. 2 is a schematic view of an apparatus for decontaminating small articles according to the present invention.

### BRIEF DESCRIPTION OF THE PREFERRED EMBODIMENTS

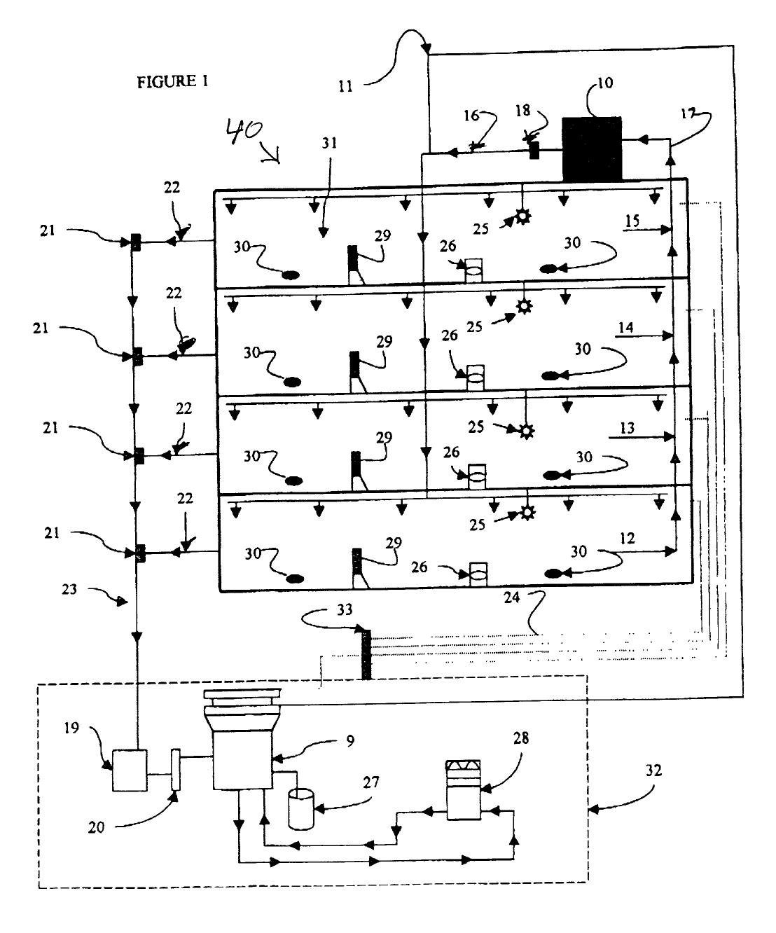

Referring to FIG. 1, there is illustrated an apparatus 32 for performing a method of decontaminating an infected building according to the present invention. Prior to the starting of the disinfecting and decontamination process with ozone a building 40 undergoes the following preparation: ozone delivery lines 24 coming from the ozone manifold 33 are placed on each floor of the building 40 to be treated. An alternate method for the distribution of ozone into the building is to connect a single ozone line 11 from the ozone generator 9 into the heating and air conditioning ductwork 16 supplying the building. Another pretreatment step is to place ozone monitoring lines 22 at the farthest point away from the ozone injection points of lines 24 and the distribution ducts of line 16. Still another treatment step is to place portable humidifiers 29 containing water or a blend of water and hydrogen peroxide or a blend of water, hydrogen peroxide solution of about 0.5 to 10%, and a pH buffer preferably a pH of 4.5 to 6.5, at strategic points in the building. If the building is equipped with its own humidifier 18, the water may also be treated with hydrogen peroxide and/or pH buffer. A further step in the treatment of the building is the placement of portable heaters 26 at strategic locations in the building. Still another treatment is the placement of portable ultraviolet lights at strategic locations in the building to aid in the treatment. Another treatment step is to elevate the temperature in the building to 80 degrees F. utilizing the buildings own heating system 10 alone or in conjunction with the heaters 26. Another treatment step is to elevate the humidity in the building 50 to 68 percent utilizing the buildings own humidifier 18 alone or in conjunction with the portable humidifiers 29. Still another treatment step is the placement of control samples of the bacteria or virus 30 to be treated. The ozone generator 9 utilizes bottled oxygen 27 as a source of feed gas. Other feed gas devices such as oxygen concentrators (not shown) and air compressors (not shown) may also be used. The ozone generator 9 may also be equipped with hose fittings (not shown) to flowing from the ozone generator 9 through ozone resistant line 11 into the discharge side of the heating and air conditioning duct work 16 and continues to be distributed through the numerous outlet vents in the building. Simultaneously the monitoring system consisting of sampling lines 22 and micro processor control valves 21 alternate sending sample readings of the ozone level through the main line 23 to the monitor 19 which in turn sends the information to the micro processor 20 which controls the output of the ozone generator 9. By operating the heating and air conditioning system 10 of the building ozone is drawn into the return air ducts 12, 13, 14, and 15 into the main return duct 17 allowing for decontamination of the duct work. Also by using ozone gas the bacteria and/or viral agent that are airborne in the building environment 31 will also be treated.

It is understood that the alternative methods of pre-treating a building depends upon its size and the location of the offices within a building. The method and apparatus illustrates a multi-floored building having a large open space such as found in post offices or terminal buildings. Less elaborate apparatuses and methods may be used for small buildings or offices where the contamination is confined.

The ozone is more effective when used in a humid or wet atmosphere. Also, the ozone is more effective at elevated temperatures and/or high pressures. Therefore, humidifiers including steam generators can be used alone or in conjunction with a heating system already installed in the building being treated.

It is already known to cover an entire building with a tarpoline and fumigate to eliminate pests. Such techniques can be used with the apparatuses of the present invention, especially in vacant buildings.

Basically the treatment of a contaminated building involves generating ozone alone or in combination with inert gases to kill a bacteria, virus or mold. Preferably, there is a humid or wet atmosphere depending on atmospheric conditions which can be supplemented by artificial means such as humidifiers and heaters.

It is preferred to provide a controlled atmosphere when possible so as to monitor the effect of the treatment through the use of a separate containment area.

The ozone can be supplemented with other anti-microbial agents such as ultraviolet agents and hydrogen peroxide.

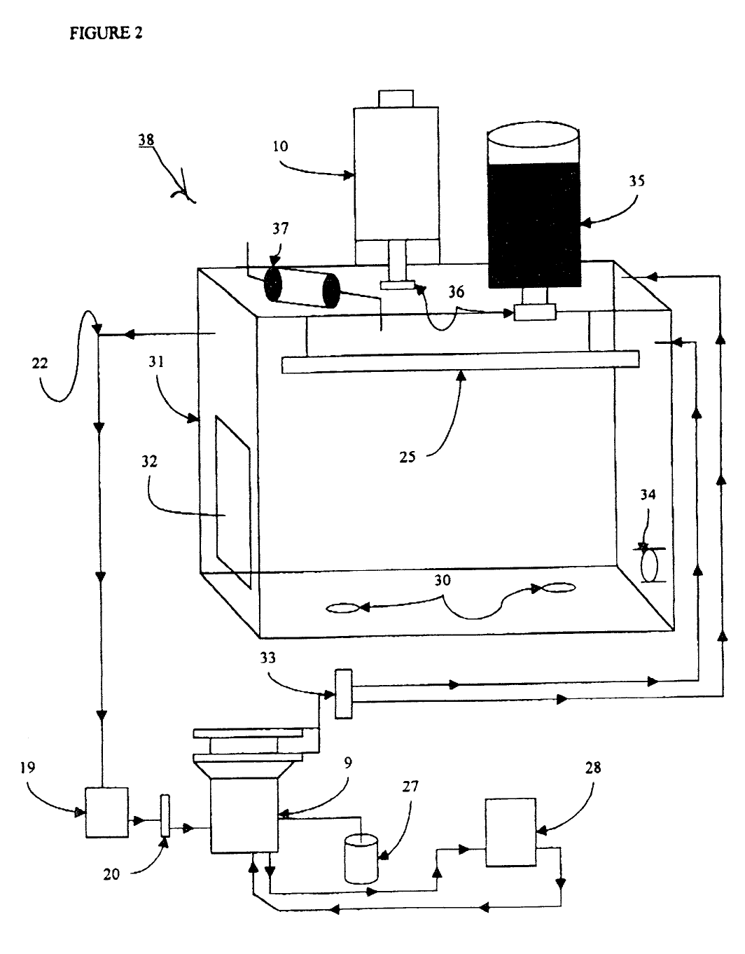

Referring to FIG. 2, there is illustrated an apparatus 38 for performing a further embodiment of the invention, namely to treat small articles. Prior to starting the disinfecting and decontamination process the step of placing item(s) to be decontaminated into a container 31 by means of the door 32. Alternatively, another treatment step is the placement of control samples of the bacteria and/or virus 30 to be treated into a separate container 31. It is preferred to elevate the temperature of the treatment container by means of a heating unit in combination with a humidifier 18 at temperatures of about 35 to 80 degrees F. and a humidity level of 50-68%, preferably about 60%. Again the solution used in the humidifier may contain combinations of water, hydrogen peroxide, and a pH buffer of 4.5-6.5. An optional step is to put the contents of the container 31 under a vacuum of approximately 10 inches utilizing a vacuum pump 37. In the event it is desirous to place the contents of the container 31 under a vacuum prior to treatment valves 36 on the combination heater/humidifier 35 and the ozone gas destruction unit 10 are closed. Another optional step is to illuminate the interior of the container 31 with ultraviolet light 25. This illumination may continue during the said process. The ozone generator can utilize bottled oxygen 27 as a source of feed gas. Other feed gas devices such as oxygen concentrators (not shown) and air compressor (not shown) may also be utilized. The ozone generator 9 utilizes a water chiller 28 for cooling of the ozone reaction chamber(s) inside the generator 9. The ozone generator may also be equipped with hose fitting (not shown) so that it may utilize water from various sources such as a coolant. The treatment consists of ozone gas flowing from the ozone generator 9 through a manifold system 33 through ozone resistant lines into the container. During the process ozone samples are taken by means of a sample line 22, which transports the samples into an ozone monitor 19. The ozone monitor is capable of sending a signal to the microprocessor 20 which in turn has the ability to raise, lower or maintain the output of the ozone generator 9. Upon completion of the ozone cycle, valves 36 are opened to allow the ozone gas into the destruction unit 10. After all pressure is relieved, valve 34 is opened to allow for air to enter the chamber thus allowing for a complete purge of the container 31.

Placing the container 31 under reduced pressure is optional since ozone alone is capable of deactivating the infectious bacteria and viral agents. The UV radiation acts synergistically to kill pathogens.

What is claimed is:

1. A method for decontaminating a building or small articles in a container infected with bacterial or viral agents or mold which comprises the steps of generating a gas comprising ozone through said building or contain with said ozone containing gas so as to be effective to kill said bacterial or viral agents or mold.

2. The method of claim 1 including providing a humid atmosphere.

3. The method of claim 1 wherein said ozone containing gas is wetted prior to treatment.

4. The method of claim 3 wherein said ozone containing gas is wetted with a solution containing hydrogen peroxide.

5. The method of claim 3 wherein said ozone containing gas is wetted with a solution containing a pH buffer.

6. The method of claim 1 wherein treatment of said building or container is at an elevated temperature.

7. The method of claim 1 including the step of exposing said bacterial or viral agents or mold to ultraviolet radiation.

8. The method of claim 1 wherein said articles in the container are treated under reduced pressure.

9. The method of claim 1 including the step of separately monitoring the treatment of the bacterial or viral agent or mold.

10. The method of claim 1 wherein said building contains air conditioning ducts and said ozone containing gas is generated into said ducts.

11. The method of claim 1 wherein anthrax spores or small pox viruses are decontaminated.

12. In combination, a building, an ozone gas generation, and means for transporting ozone throughout said building to deactivate or decontaminate bacterial or viral agents or mold which is in said building.

13. The combination of claim 13 wherein said means for transporting ozone comprise air conditioning ducts.

14. The combination of claim 13 including means for providing UV radiation.

15. An apparatus for decontaminating small articles which comprises a housing having an entry port, means for transporting an ozone containing gas into said housing and means for providing UV radiation whereby any pathogens or mold within said housing is exposed to UV radiation and ozone.

16. The apparatus of claim 16 including means for reducing pressure within said housing.

17. The apparatus of claim 16 including means for elevating the temperature within said housing.

18. The apparatus of claim 16 including means for raising the humidity in said housing.

19. The apparatus of claim 16 including a means for monitoring the ozone within said apparatus.
